(19) United States
(12) Patent Application Publication
(10) Pub. No.: US 2006/0172299 A1
(43) Pub. Date: Aug. 3, 2006

(54) Methods for the synthesis of mucleic acids

(76) Inventor: Guido Krupp, Gnutz (DE)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 10/526,487

(22) PCT Filed: Sep. 2, 2003
(86) PCT No.: PCT/EP03/09756

§ 371 Date: Nov. 8, 2005

(30) Foreign Application Priority Data
Sep. 4, 2002 (DE) 102 40 868.8

(51) Int. Cl. C12Q 1/68; C12P 19/34

(52) U.S. Cl. 435/6; 435/91.2

(57) ABSTRACT

The invention relates to a method for the synthesis of nucleic acids, where incubation of a polymerase, a nucleic acid that can be used as a template for the polymerase, NTPs and Mn²⁺ is performed under conditions that enable the synthesis of a nucleic acid strand and the method is characterised by conditions that comprise a molar ratio of Mn²⁺/NTP of not more than 0.7.

### CROSS-REFERENCE TO RELATED APPLICATIONS

This application is a national phase application under 35 U.S.C. § 371 of International Application Number PCT/EP2003/009756, filed Sep. 2, 2003, the disclosure of which is hereby incorporated by reference in its entirety, and claims the benefit of German Patent Application Number 102 40 868.8, filed Sep. 4, 2002.

### BACKGROUND OF THE INVENTION

The present invention relates to improved methods for the synthesis of nucleic acids, comprising the incubation of a polymerase, a nucleic acid that can serve as a template for the polymerase, NTPs and Mn²⁺ under conditions enabling the synthesis of a nucleic acid strand wherein these conditions are characterised by comprising a molar ratio of Mn²⁺/NTP of not more than 0.7.

The invention relates in particular to methods for the preparation of RNA, wherein DNA is used as a template and wherein at least a 1000-fold amplification rate is achieved. Further, the present invention relates to kits which comprise the components required for performing the methods according to the invention.

In vitro amplification of nucleic acids is necessary to perform a number of methods of molecular biology, for example cloning, sequence analysis, in vitro expression etc. Accordingly, methods were developed which permit in vitro synthesis of nucleic acids. In general, these procedures can be distinguished on the basis of the reaction product, which is either DNA or RNA.

In vitro transcription is a method to synthesise RNA, usually utilising double-stranded DNA as a template. In this method, isolated components of the cellular transcription machinery (RNA polymerase and NTPs) are used for an enzymatic reaction, performed in a reaction tube. It is assumed, that a complex of Mg²⁺ and NTP is the substrate for the synthesis reaction. Therefore, Mg²⁺ is an essential component of the reaction and it is usually added in excess in relation to the NTP concentrations (Milligan and Uhlenbeck, Methods in Enzymology, Vol. 180 (1989), 51-62; and Wyatt et al., Biotechniques, Vol. 11 (1991), 764-769).

To optimise the amplification rate of the in vitro transcription, e.g., U.S. Pat. No. 5,256,555 suggests the use of a total nucleotide concentration of more than 16 mM. At the same time, Mg²⁺ that is essential for the reaction, should be used in a concentration that is not higher than 10% above the total concentration of all nucleotides. Furthermore, inorganic pyrophosphatase should be present in the reaction.

The best known method for DNA synthesis is the polymerase chain reaction (PCR), developed by Kary Mullis in the mid-eighties (see, Saiki et al., Science, Vol. 230 (1985), 1350-1354; and EP 201 184).

During the PCR reaction, single stranded primers (oligonucleotides with a chain-length of usually 12 to 24 nucleotides) anneal to a complementary, single stranded DNA sequence. The primers are elongated by a DNA polymerase and the deoxyribonucleoside triphosphates (dNTPs, i.e., dATP, dCTP, dGTP, dTTP), to obtain a double stranded DNA. The double stranded DNA is separated by heating into single strands. The temperature is reduced sufficiently to permit a new step of primer annealing. Again, primers are elongated by DNA polymerase to obtain a second DNA stand.

Repetition of the steps described above enables the exponential amplification of the DNA template strands. This is achieved by adjusting the reaction conditions such that almost each reaction cycle results in the conversion of each DNA single strand to a double strand, subsequently separated again in two single strands which are used again as template for further strands.

If a reverse transcription reaction is performed prior to this method, wherein an mRNA is converted to a single stranded DNA (the so-called cDNA) by means of an RNA-dependent DNA polymerase, then the PCR reaction can directly be applied for amplification of nucleic acids starting from an RNA sequence (see, EP 201 184).

In addition to the mentioned basic reaction schemes, a multitude of alternatives have been developed, that can be distinguished based on the starting materials (RNA, DNA, single or double stranded) and the reaction product (amplification of specific RNA or DNA sequences from one sample, or the amplification of all sequences).

Both, the patents DE 101 43 106.6 and DE 102 24 200.3 describe methods for the amplification of ribonucleic acids, that comprise a combination of individual steps of the PCR reaction and a transcription reaction.

Despite the above advances, there is still a need for further improvement of methods for the synthesis of nucleic acids, in particular for methods that permit a high synthesis rate and synthesis yield in combination with a low consumption of chemicals.

This problem has now been solved by providing a method for the synthesis of nucleic acids, comprising the incubation of a polymerase, a nucleic acid that can serve as a template for the polymerase, NTPs and Mn²⁺ under conditions that permit the synthesis of a nucleic acid strand, characterised in that the conditions comprise a molar ratio of Mn²⁺/NTP of not more than 0.7.

### SUMMARY OF THE INVENTION

The present invention provides a method for the synthesis of nucleic acids, comprising incubating a polymerase, a nuleic acid that can serve as a template for the polymerase, NTPs and Mn²⁺ under conditions that permit the synthesis of a nucleic acid strand, where the conditions comprise a molar ratio of Mn²⁺/NTP of not more than 0.7. The present invention also provides a kit for the synthesis of nucleic acids that comprises a polymerase, NTPs and Mn²⁺, in one container or in several separate containers.

The present invention provides a method for the synthesis of nucleic acids, comprising the incubation of a polymerase, a nucleic acid that can serve as a template for the polymerase, NTPs and Mn²⁺ under conditions that permit the synthesis of a nucleic acid strand, characterised in that the conditions comprise a molar ratio of Mn²⁺/NTP of not more than 0.7. The present invention further provides said method for the synthesis of nucleic acids, characterised in that the polymerase is an RNA polymerase, preferably a DNA dependant RNA polymerase that needs a DNA template having a promoter to synthesize RNA. The present invention further provides said method for the synthesis of nucleic acids, characterised in that the molar ratio of Mn²⁺/NTP is between 0.2 and 0.6, preferably 0.3 to 0.5. The present invention further provides said method for the synthesis of nucleic acids, characterised in that the total NTP concentration is between 4 mM and 24 mM. The present invention further provides said method for the synthesis of nucleic acids, characterised in that the Mn²⁺ concentration is at least 3 mM, preferably at least 3.5 mM or at least 4 mM. The present invention further provides said method for the synthesis of nucleic acids, characterised in that the Mn²⁺ concentration is between 4 mM and 17 mM. The present invention further provides said method for the synthesis of nucleic acids, characterised in that the polymerase is a T7 RNA polymerase, a T3 RNA polymerase or an SP6 RNA polymerase. The present invention further provides said method for the synthesis of nucleic acids, characterised in that DNA or RNA is used as the nucleic acid that can serve as a template for the RNA polymerase. The present invention further provides said method for the synthesis of nucleic acids, characterised in that DNA or RNA is used as the nucleic acid that can serve as a template for the RNA polymerase and this nucleic acid is present in an amount of at least 0.1 picogram (or 0.2 attomol, respectively) or in a concentration of at least 10 femtomolar. The present invention further provides said method for the synthesis of nucleic acids, characterised in that ATP, UTP, CTP and/or GTP are used as NTPs. The present invention further provides said method for the synthesis of nucleic acids, characterised in that also dNTPs can be used. The present invention further provides said method for the synthesis of nucleic acids, characterised in that dATP, dTTP, dCTP and/or dGTP are used as dNTPs. The present invention further provides said method for the synthesis of nucleic acids, characterised in that the NTPs or dNTPs can be used as derivatives, for example as biotinylated derivatives or coupled to a fluorescence label. The present invention further provides said method for the synthesis of nucleic acids, characterised in that an amplification rate of at least 1000-fold, preferably at least 2000-fold, is achieved.

The present invention provides a kit for the synthesis of nucleic acids that comprises a polymerase, NTPs and Mn²⁺, in one container or in several separate containers. The present invention further provides said kit for the synthesis of nucleic acids, characterised in that the polymerase is a DNA dependant RNA polymerase that needs a DNA template having a promoter to synthesize RNA, wherein preferably a T7 RNA polymerase, a T3 RNA polymerase or a SP6 RNA polymerase is used. The present invention further provides said kit for the synthesis of nucleic acids, characterised in that it comprises ATP, UTP, CTP and/or GTP as NTPs. The present invention further provides said kit for the synthesis of nucleic acids, characterised in that it further comprises dNTPs. The present invention further provides said kit for the synthesis of nucleic acids, characterised in that it comprises dATP, dTTP, dCTP and/or dGTP as dNTPs. The present invention further provides said kit for the synthesis of nucleic acids, characterised in that it comprises NTPs or dNTPs in the form of derivatives, for example as biotinylated derivatives or coupled to a fluorescence label. The present invention further provides said kit for the synthesis of nucleic acids, characterised in that it further comprises instructions for performing a method for the synthesis of nucleic acids (e.g., a method for the synthesis of nucleic acids, comprising the incubation of a polymerase, a nucleic acid that can serve as a template for the polymerase, NTPs and Mn²⁺ under conditions that permit the synthesis of a nucleic acid strand, characterised in that the conditions comprise a molar ratio of Mn²⁺/NTP of not more than 0.7).

### BRIEF DESCRIPTION OF THE DRAWINGS

FIG. 1 In vitro transcription using different concentrations of Mn²⁺ and Mg²⁺ and NTPs with a total concentration of 16 mM. Determination of the optimal Mn²⁺/NTP ratio and comparison with Mg²⁺.

FIG. 2 Determination of the optimal NTP concentration, using various Mn²⁺/NTP ratios.

FIG. 3 Determination of the amplification rate.

### DETAILED DESCRIPTION OF THE INVENTION

Surprisingly, the method of the present invention was able to show that already the choice of the above mentioned molar ratio of Mn²⁺/NTPs can result in a significantly increased synthesis rate of the polymerase. At the same time, the method according to the invention is more cost-efficient, because lower NTP concentrations are used. Therefore, a high amplification rate can be achieved by simple and cost-efficient means.

In the context of the present invention, the term “molar ratio of Mn²⁺/NTP” is used to provide a numerical value for the quotient of the molar concentration of Mn²⁺ in relation to the total molar concentration of all NTPs.

In an especially preferred embodiment of the method according to the invention, the synthesis conditions are chosen that the a molar ratio of Mn²⁺/NTP is between 0.2 and 0.6, preferably between 0.3 and 0.5.

Using the molar ratios according to the invention may result in a total NTP concentration between 4 mM and 24 mM; the use of 4 different NTPs results in concentrations between 1 mM and 6 mM for each NTP.

This provides a preferable Mn²⁺ concentration range between 0.8 mM (molar ratio of 0.2 and total NTP concentration of 4 mM) and 14.4 mM (molar ratio of 0.6 and total NTP concentration of 24 mM).

Any polymerase can be used in the method according to the invention. However, the use of an RNA polymerase is preferred for all embodiments of the invention, and in particular a DNA dependant RNA polymerase that needs a DNA template having a promoter for the synthesis of RNA. For example a T7 RNA polymerase, a T3 RNA polymerase or an SP6 RNA polymerase may be used.

The RNA polymerase can either be an RNA-dependent or a DNA-dependent polymerase. Most naturally occurring DNA-dependent RNA polymerases can also use RNA as a template, if present in a suitable structure (see Konarska, M. M. and Sharp, P: A:, Cell, Vol. 57 (1989),423-431; and Konarska, M. M and Sharp, P. A. Cell, Vol. 63 (1990), 609-618).

The polymerase and the template nucleic acid have to be compatible. For example, the template nucleic acids used by an RNA polymerase have to comprise certain sequences or structures that are recognised by the RNA polymerase, and that permit initiation of the synthesis. Preferably, DNA is used as a template for the RNA polymerase. The corresponding DNA may contain a promoter region, that is recognised by the RNA polymerase and that is used for the start of the synthesis. Alternatively, the DNA may contain other structures, which are recognised by the RNA polymerase and allow initiation of the synthesis. Such alternative structures are described in Krupp (Nucleic Acid Res., Vol. 17 (1989), 3023-3036) and in Kuhn et al. (Nature, Vol. 344 (1990), 559-562).

Because the method according to the present invention results in a very high amplification rate, the template nucleic acid can be used in very low concentration. For example, the template can be DNA or mRNA and it is used in an amount of at least 0.1 picogram, or 0.2 attomol, respectively. In a reaction volume of 20 μl this results in a minimum concentration of 10 femtomolar.

The reaction mix has to contain NTPs. RNA polymerase is usually used in combination with ATP, UTP, CTP, and GTP. In the conventional transcription reaction according to the state of the art, all mentioned NTPs are used in a reaction. However, it can be desired or useful, to use only one or a few of the NTPs.

Alternatively, or in addition to the NTPs, dNTPs can be used in the method according to the invention combined with the use of an RNA or a DNA polymerase. This way of proceeding has the special advantage that the transcript has complete or partial DNA characteristics, i.e., it becomes nuclease resistant and serves as a better template for the RNA polymerase. Commonly used dNTPs are dATP, dTTP, dCTP and/or dGTP.

All or some of the NTPs and/or of the dNTPs can be used as modified compounds or derivatives. According to the state of the art, commonly used derivatives include coupling to biotin or to a fluorescence label, e.g., for simplifying the detection of the synthesis products.

The reaction time and further reaction conditions (temperature, pH and so forth) are easily chosen by one skilled in the art, and they depend on the polymerase used and the expected amplification rate. The incubation time, for example, can range between 1 h and 24 h; however, preferably between 4 h and 16 h. If the T7 RNA polymerase is used, an incubation temperature between 30° C. and 45° C. is the obvious choice.

The method according to the invention surprisingly provides an improved amplification rate. In the context of the present invention, the term “amplification rate” means the ratio of the amount of synthetically produced nucleic acids in relation to the amount of the input template. The method according to the invention permits an amplification rate of at least 1000, preferably at least 2000. Under optimal reaction conditions, even an amplification rate of 2500 was achieved.

The methods according to the invention can be used for a multitude of applications. For example, the improved methods for the production of nucleic acids can be used in the methods for amplification of ribonucleic acids as described in DE 101 43 106.6 and DE 102 24 200.3. The nucleic acids obtained according to the method of the invention can be coupled to a chip and used as probes. The methods can be used for the in vitro transcription, for the analysis of interactions with nucleic acid binding factors, as aptamers for specific binding of molecules, as ribozymes, etc.

Finally, the present invention relates to kits for the synthesis of nucleic acids, which comprise one or more containers, containing a polymerase, NTPs, dNTPs and/or their derivatives (for example, NTPs or dNTPs that are biotinylated or coupled to a fluorescence label) and Mn²⁺. Preferably, the polymerase is an RNA polymerase, preferably a DNA dependant RNA polymerase that needs a DNA template having a promoter to synthesize RNA. Especially preferred is the use of the T7 RNA polymerase, the T3 RNA polymerase, or the SP6 RNA polymerase.

Preferably, such kits further contain instructions for performing one of the methods according to the invention. Such instructions or manuals will describe in full detail the amounts of the different reaction components that have to be mixed to obtain optimal synthesis results.

### EXAMPLES

### Example 1

In this example, the transcription rates of the RNA polymerase were determined, dependent on various concentrations of Mn²⁺ as well as Mg²⁺.

For this purpose, a 20 μl reaction volume, the components 40 mM Tris-HC1 (pH 8), 10 mM DTT, 2 mM spermidine, 0.01% Triton X-100, 10 ng of a nucleic acid template (plasmid pTRI-Xef), 10 U RNasin (RNase-inhibitor), 40 U T7 RNA polymerase, 4 mM NTPs (each; resulting in a total of 16 mM), and Mn²⁺ or Mg²⁺ in a concentration ranging from 4 mM to 10 mM were pipetted together and were incubated for 16 h.

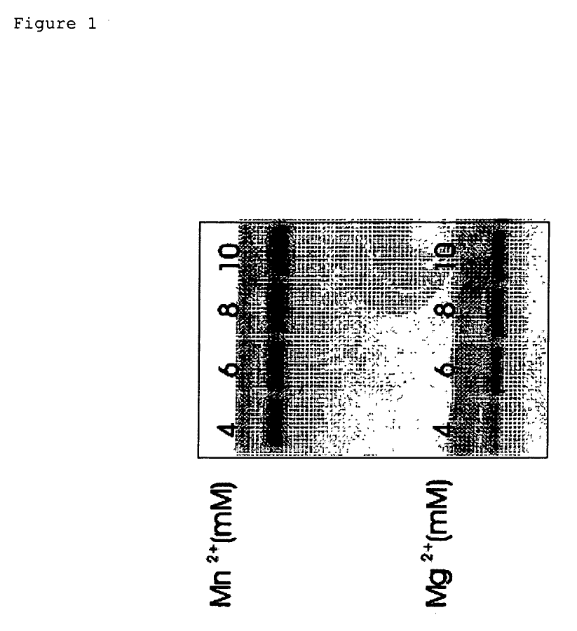

Aliquots of 5 μl were withdrawn and separated by means of electrophoresis on a 1% agarose gel, and photographed after staining with ethidium bromide. The result is shown in FIG. 1. Dependant on the concentrations of Mn²⁺ and Mg²⁺, respectively, the resulting ratios of Mn²⁺/NTPs vary between 0.25 and 0.625.

FIG. 1 clearly shows for all reaction conditions, that the presence of Mn²⁺ resulted in a 2+better amplification rate, as compared with Mg

### Example 2

The aim of this example was to determine the optimal NTP concentration, dependent on the Mn²⁺/NTP ratio.

For this purpose, a series of in vitro transcription reactions were performed, as outlined in example 1. The concentration of NTPs ranged from 2 mM to 10 mM for each NTP, and the MnCl₂ concentrations were between 2.4 mM and 24 mM, resulting in a Mn²⁺/NTP ratio between 0.3 and 0.6.

The amount of transcript obtained (in ng) was determined by staining the gel with ethidium bromide and the use of an RNA dilution series as reference.

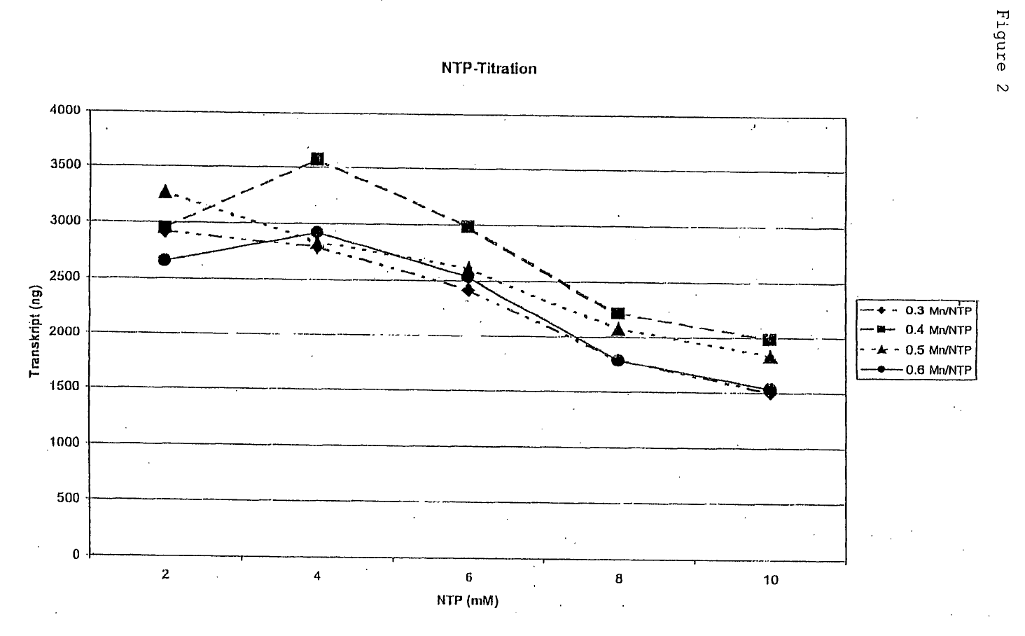

The result is summarised in FIG. 2 and shows, that already at a concentration of 4 mM of each NTP (total concentration of NTPs was 16 mM) resulted in a maximal synthesis rate. The best results were obtained using a combination of 4 mM of each NTP and 6.4 mM MnCl₂ (corresponds to a ratio of 0.4)

### Example 3

In this example, the amplification rate was determined in relation to the incubation time. For this purpose first of all, an in vitro transcription reaction as described in example 1 was set up, wherein 4.8 mM MnCl₂ and 4 mM NTP (total of 16 mM) were used (corresponds to a Mn²⁺/NTP ratio of 0.3).

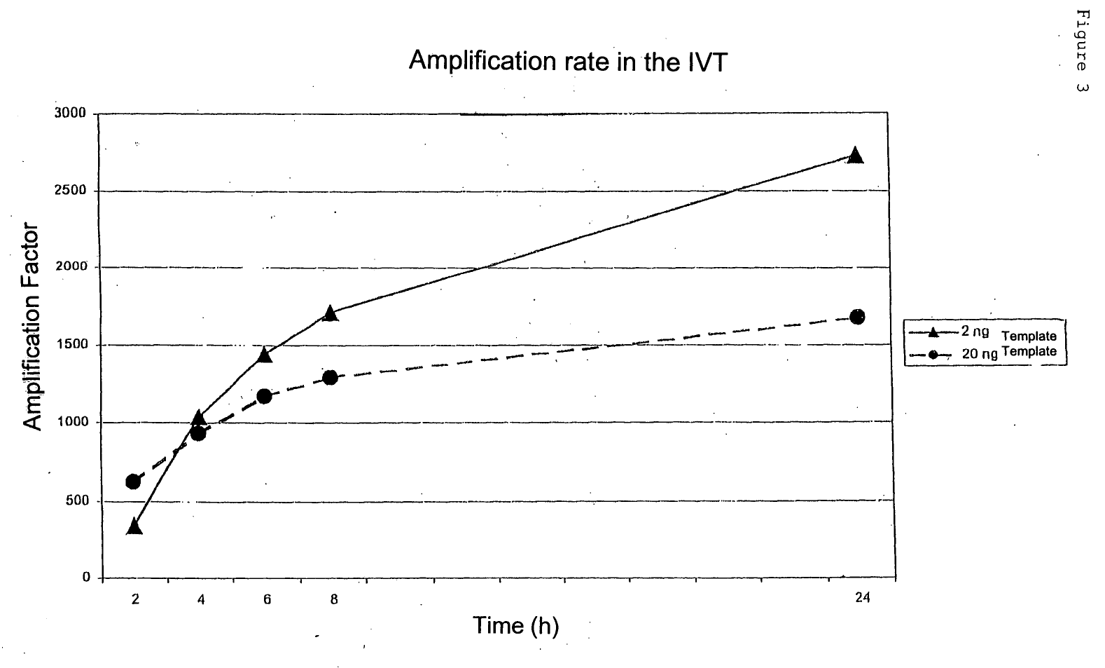

At the time points indicated in FIG. 3, aliquots of 5 μl were withdrawn and analysed on a 1% native agarose gel. The results are shown in FIG. 3 and they reveal, that an amplification factor of more than 1500 was obtained in all reaction conditions.

1.-27. (canceled)

28. A method for the synthesis of nucleic acids, comprising incubating a polymerase, a nuleic acid that can serve as a template for the polymerase, NTPs and Mn²⁺ under conditions that permit the synthesis of a nucleic acid strand, wherein the conditions comprise a molar ratio of Mn²⁺/NTP of not more than 0.7.

29. The method according to claim 28, wherein the polymerase is an RNA polymerase.

30. The method according to claim 28, wherein the polymerase is a DNA dependant RNA polymerase that needs a DNA template having a promoter to synthesize RNA.

31. The method according to claim 28, wherein the molar ratio of Mn²⁺/NTP is between 0.2 and 0.6.

32. The method according to claim 28, wherein the molar ratio of Mn²⁺/NTP is between 0.3 and 0.5.

33. The method according to claim 28, wherein the total NTP concentration is between 4 mM and 24 mM.

34. The method according to claim 28, wherein the Mn²⁺ concentration is at least 3 mM.

35. The method according to claim 28, wherein the Mn²⁺ concentration is at least 3.5 mM.

36. The method according to claim 28, wherein the Mn²⁺ concentration is at least 4 mM.

37. The method according to claim 28, wherein the Mn²⁺ concentration is between 4 mM and 17 mM.

38. The method according to claim 28, wherein the polymerase is a T7 RNA polymerase, a T3 RNA polymerase or an SP6 RNA polymerase.

39. The method according to claim 28, wherein DNA or RNA is used as the nucleic acid that can serve as a template for the polymerase.

40. The method according to claim 28, wherein DNA or RNA is used as the nucleic acid that can serve as a template for the polymerase and this nucleic acid is present in an amount of at least 0.1 picogram or in a concentration of at least 10 femtomolar.

41. The method according to claim 28, wherein one or more of ATP, UTP, CTP and GTP are used as NTPs.

42. The method according to claim 28, wherein also dNTPs can be used.

43. The method according to claim 42, wherein one or more of dATP, dTTP, dCTP and dGTP are used as dNTPs.

44. The method according to claim 42, wherein the NTPs or dNTPs comprise derivatives of NTPs or dNTPs.

45. The method according to claim 28, wherein an amplification rate of at least 1000-fold is achieved.

46. The method according to claim 28, wherein an amplification rate of at least 2000-fold is achieved.

47. A kit for the synthesis of nucleic acids that comprises a polymerase, NTPs and Mn²⁺, in one container or in several separate containers.

48. The kit according to claim 47, wherein the polymerase is a DNA dependant RNA polymerase that needs a DNA template having a promoter to synthesize RNA.

49. The kit according to claim 47, wherein the polymerase is a T7 RNA polymerase, a T3 RNA polymerase or a SP6 RNA polymerase.

50. The kit according to claim 47, comprising one or more of ATP, UTP, CTP and GTP as NTPs.

51. The kit according to claim 47, further comprising dNTPs.

52. The kit according to claim 51, comprising one or more of dATP, dTTP, dCTP and dGTP as dNTPs.

53. The kit according to claim 51, wherein the NTPs or dNTPs comprise derivatives of NTPs or dNTPs.

54. The kit according to claim 47, further comprising instructions for performing synthesis of nucleic acids.
